(19) United States
(12) Patent Application Publication
(10) Pub. No.: US 2007/0289032 A1
(43) Pub. Date: Dec. 13, 2007

(54) AGROBACTERIUM TRANSFORMATION OF GUAR

(75) Inventors: Richard Dixon, Ardmore, OK (US); Sanchita Vaghchhipawala, Ardmore, OK (US)

Correspondence Address: FULBRIGHT & JAWORSKI L.L.P., 600 CONGRESS AVE., SUITE 2400, AUSTIN, TX, 78701, US

(73) Assignee: SAMUEL ROBERTS NOBEL FOUNDATION

(21) Appl. No.: 11/757,164

(22) Filed: Jun. 1, 2007

Related U.S. Application Data
(60) Provisional application No. 60/810,252, filed on Jun. 2, 2006.

(51) Int. Cl. C12N 15/82; A01H 5/00; A01H 1/00

(52) U.S. Cl. 800/278; 800/294; 800/279; 800/298

(57) ABSTRACT

The invention provides methods for transforming guar plants with Agrobacterium. The invention allows creation of transgenic guar plants with improved traits.

This application claims the priority of U.S. Provisional Patent Application 60/810,252, filed Jun. 2, 2006, the disclosure of which is incorporated herein by reference in its entirety.

### BACKGROUND OF THE INVENTION

1. Field of the Invention

The present invention relates generally to the fields of botany and molecular biology. More specifically, the invention relates to guar plant transformation using Agrobacterium-mediated gene transfer.

2. Description of the Related Art

Guar (Cyamopsis tetragonoloba), also known as cluster bean, is a large seeded sub-tropical legume. It produces a cell wall storage polysaccharide gum commonly known as guar gum. Structurally, guar gum comprises a long chain β1→4 linked D-mannan backbone with single unit 1→6 α-linked D-galactopyranosyl substituents. Guar is slowly gaining the status of a commercial crop because of the versatility of its gum as a viscosity enhancer in the food industry, and additional uses in the paper, cosmetics, mining, oil and explosive industries (Sainy and Paroda, 1984). There is ample scope to exploit this crop biotechnologically. Apart from being a source of gum, guar has been widely used as a rotation crop, green manure, cattle feed and vegetable (young pods) in India and Pakistan. This drought tolerant annual legume was introduced to the U.S. in 1903 from India. It is cultivated in Northern Texas and South Western Oklahoma.

The world market for guar gum is estimated to be around 150,000 tons/year, and is mainly fed by India and Pakistan. U.S. consumption is estimated to be around 40,000 tons/year. The demand is almost constant whereas the supply depends on the monsoon rains in western India and Pakistan, resulting in volatility in production and consequently in price. Hence there exists a need for developing a high yielding, drought tolerant variety with better gum quality which could be cultivated in a cost effective manner in the United States.

One of the most important constraints towards biotechnological manipulation of guar is its poor ability to respond to in vitro genetic transformation and subsequent regeneration of the transformed tissue. Like most other large seeded legumes, Guar is recalcitrant to transformation, and regeneration in vitro is highly genotype specific with most of the cultivated varieties in the United States being unable to root in vitro. Only a few reports are available on guar plant tissue culture establishment and growth (Bansal et al., 1994; Prem et al., 2005; Ramulu and Rao, 1993a; Ramulu and Rao, 1993b), protoplast isolation and culture (Saxena and Gill, 1986; Saxena et al., 1986), or gene transfer (Joersbo et al., 1999; Joersbo et al., 2001). The varieties which are regenerable are not highly transformable, and the varieties which are transformable are not capable of rooting in culture. Joersbo et al reported a transformation procedure for an Indian cultivar of unknown origin and two other American cultivars, but the plants were not capable of developing any roots in vitro, resulting in the need for time consuming (up to a total of 10 weeks) grafting on a non-transformed stock tissue (Joersbo et al., 1999).

### SUMMARY OF THE INVENTION

In one aspect, the invention provides a method of transforming a guar plant comprising: (a) obtaining an explant from a guar seedling; (b) contacting the explant with a recombinant Agrobacterium tumefaciens strain containing a DNA of interest to transform at least a first cell of the explant with the DNA of interest; (c) culturing the explant to induce formation of roots; (d) allowing roots to form from the explant; and (e) cultivating the explant under plant growth conditions to produce a transgenic guar plant comprising the DNA of interest. In certain embodiments, the explant may be a cotyledon explant, and may be a cotyledonary node. The DNA of interest may comprise a selectable marker, and the explant may be contacted with a selective agent following transforming the first cell with the DNA of interest. In one embodiment, the guar explant is from cultivar Lewis or Kinman. In another embodiment, the Agrobacterium tumefaciens strain is C58C1.

In particular embodiments of the invention, culturing the explant to induce formation of roots may comprise contacting the explant with Shoot Initiation Medium (SIM) and/or contacting the explant with Shoot Elongation Medium (SEM) and allowing shoots to form. Shoots may be, for example, about 2-5 mm in length or about 5-10 mm in length. Culturing the explant to induce formation of roots may comprise infection with A. rhizogenes or culturing in root induction medium. Culturing the explant to induce formation of roots may comprise culturing in Farhaeus medium for about 4 to 6 weeks. In one embodiment of the invention, cultivating the explant under plant growth conditions comprises transferring the explant to soil. In specific embodiments, the A. rhizogenes is strain ARqua1.

In further embodiments of the invention, an explant may be obtained from about 7 to 9 days post-germination. Step (b) may comprise co-cultivating the explant and Agrobacterium tumefaciens strain for about 2 days. In one embodiment, the explant is contacted with the Shoot Induction Medium for about 10 to 14 days. The Shoot Induction Medium may comprise a selective agent. In another embodiment, the explant is contacted with Shoot Elongation Medium (SEM) for about 7 to 14 days Culturing the explant to induce formation of roots may comprise culturing the explant in Root Induction Medium.

In a method of the invention, the DNA of interest may confer a trait selected from the group consisting of herbicide tolerance, an insect resistance, disease resistance, pest resistance, improved nutritional quality, modified carbohydrate metabolism and modified lipid metabolism. In one embodiment, the DNA of interest encodes β-mannan synthase or α-galactosyltransferase.

Another aspect of the invention is a guar plant generated according to the methods provided herein, including transgenic progeny thereof. Plant parts, including cells of such a plant are also provided, as well as seeds.

In yet another aspect, the invention provides a method of producing food or feed, comprising (a) obtaining a plant or part thereof made according to the invention; and (b) producing food or feed from the plant or part thereof.

The invention also provides a method of breeding a plant according to the invention, comprising, (a) obtaining a plant made by a method of the invention; and (b) crossing the plant with a second plant to produce progeny that contain the DNA of interest.

It is contemplated that any method or composition described herein can be implemented with respect to any other method or composition described herein. The use of the word “a” or “an” when used in conjunction with the term “comprising” in the claims and/or the specification may mean “one,” but it is also consistent with the meaning of “one or more,” “at least one,” and “one or more than one.” “About” means plus or minus 5% of the stated value.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein:

FIGS. 1A-D—Guar regeneration (stage 1). FIG. 1A shows shoot bud initiation. FIG. 1B shows shoot elongation. FIG. 1C shows regenerated plant. FIG. 1D shows regenerated plants prior to rooting.

FIGS. 2A-C—Guar regeneration (stage 2). FIG. 2A shows 8 week old plants in rooting medium. FIG. 2B shows 10 week old plantlets in rooting medium. FIG. 2C shows regenerated plants in tubes prior to green house.

FIGS. 3A-D—Histochemical Gus assay in 4-6 week old callus/roots. FIG. 3A shows X-gluc stained 4 week old callus. FIG. 3B shows X-gluc stained 4 week old transformed shoot. FIG. 3C shows 5 week old shoot showing Gus staining at the leaf tip. FIG. 3D shows a leaf tip showing Gus staining along the leaf margin.

FIGS. 4A-D —Agrobacterium rhizogenes mediated rooting in regenerated guar shoots. FIG. 4A shows root initiation. FIG. 4B shows regenerated root-stem junction.

FIG. 4C shows cross section of regenerated root. FIG. 4D shows cross section of root node showing the vascular connection with the stem.

FIGS. 5A-B—Establishment of root in soil. FIG. 5A shows 7 week old mature root.

FIG. 5B shows regenerated plant with root in soil.

FIG. 6—Transformation protocol.

### DETAILED DESCRIPTION OF THE INVENTION

With the emerging need for biotechnological manipulation of legumes (especially the large seeded legumes), many groups of scientists throughout the world are working on improving genetic transformation techniques. In recent years improved transformation protocols have been reported for Vigna radiata (commonly known as mung bean) (Bansal et al., 1994), cowpea (Popelka et al., 2006), as well as soybean. Like other large seeded legumes, guar is also transformation-recalcitrant and the success in transformation depends on a number of factors. Joersbo et al. (1999) reported guar transformation methodologies in the economically important American cultivars Lewis and Santa Cruz, but the transformation efficiency was not very high (0.8%), and, more important, the protocol was time consuming and required a grafting step for rooting (Joersbo et al., 1999). Although the authors concluded that the protocol was optimized to function irrespective of the cultivar, the inventors found that the cultivar and the Agrobacterium strain play a crucial role in determining the overall transformation efficiency as determined by transient GUS activity. In the transformation process, in vitro rooting is the greatest hurdle in the American cultivars and the inventors were able to induce roots in vitro which were morphologically and functionally normal and supported the plant in the soil. Prem et al. (2005) reported a regeneration and rooting protocol for a number of Indian cultivars but it is not known whether those genotypes are amenable to genetic transformation.

Herein are described new protocols for guar transformation, exemplified for two high yielding, disease resistant, drought tolerant American cultivars (Lewis and Kinmann). Three different aspects of the transformation/regeneration process are addressed: first, determining the best Agrobacterium strain for optimum transformation efficiency; second, optimizing the shoot regeneration method for subsequent in vitro rooting; and third, developing rooting procedures for the regenerated shoots using either Agrobacterium rhizogenes or growth regulators. Five different Agrobacterium tumefaciens strains have been utilized to compare the efficacy of gene transfer in Lewis and Kinman. Among the strains LBA4404, AGL1, EHA 105, GV3101 (Holsters et al., 1980) and C58C1 (Hamilton and Fall, 1971) harboring a binary vector with GUS and kanamycin resistance genes, C58C1 resulted in the highest number of GUS-positive transformed calli or shoots. For shoot regeneration, different explants were used, e.g., cotyledon, cotyledonary node and embryo. Shoots induced in the cotyledonary explants in the protocol of Joersbo et al. (1999) took up to six weeks to completely regenerate and an additional two weeks for grafting on a non-transformed seedling stock (Joersbo et al., 1999). In the present protocol, the cotyledonary node explants regenerated within two weeks and 5-10 mm long shoots formed roots within four weeks in response to Agrobacterium rhizogenes. Rooting induced by growth regulators, e.g., IBA and NAA took a longer time (up to eight weeks), but in both the cases the roots were normal morphologically and physiologically and subsequent transfer to soil led to a normal healthy root system. Details of the invention are presented below.

### I. GUAR

A. General Information

Guar, or cluster bean, (Cyamopsis tetragonoloba (L.) Taub) is a drought-tolerant annual legume that was introduced into the United States from India in 1903. Commercial production of guar in the United States began in the early 1950s and has been concentrated in northern Texas and southwestern Oklahoma. The major world suppliers are India, Pakistan and the United States, with smaller acreages in Australia and Africa. In the early 1980s, Texas growers were planting about 100,000 acres annually. They harvested about half of the planted acreage and plowed the rest under as green manure.

Unlike the seeds of some other legumes, the guar bean has a large endosperm. This spherical-shaped endosperm contains significant amounts of galactomannan gum (19 to 43% of the whole seed), which forms a viscous gel in cold water. Guar gum is the primary marketable product of the plant. India and Pakistan export much of their guar crop to the United States and other countries in the form of partially processed endosperm material. World demand for guar has increased in recent years, leading to crop introductions in several countries.

Like other legumes, guar is an excellent soil-building crop with respect to available nitrogen. Root nodules contain nitrogen-fixing bacteria, and crop residues, when plowed under, improve yields of succeeding crops. In Asia, guar beans are used as a vegetable for human consumption, and the crop is also grown for cattle feed and as a green manure crop. In the United States, highly refined guar gum is used as a stiffener in soft ice cream, a stabilizer for cheeses, instant puddings and whipped cream substitutes, and as a meat binder. Most of the crop in the United States, however, is grown for a lower grade of guar gum, which is used in cloth and paper manufacture, oil well drilling muds, explosives, ore flotation, and a host of other industrial applications.

Guar gum consists of long branching polymers of mannose and galactose in a 2:1 ratio. After extraction of the gum, guar meal contains approximately 35% protein, which is about 95% digestible. The seed protein is low in methionine, like most legumes. Enough gum remains in the meal to make it an excellent feed pelleting material. Toasting improves its palatability to livestock and helps remove a trypsin inhibitor for non-ruminants.

Guar is an upright, coarse-growing summer annual legume known for its drought resistance. Its deep tap roots reach moisture deep below the soil surface. Most of the improved varieties of guar have glabrous (smooth, not hairy) leaves, stems and pods. Plants have single stems, fine branching or basal branching (depending on the variety) and grow to be 18 to 40 in. tall. Racemes are distributed on the main stem and lateral branches. Pods are generally 1½ to 4 in. long and contain 5 to 12 seeds each. Seeds vary from dull-white to pink to light gray or black and range from 900 to 1,600 seeds/oz.

Guar tolerates high temperatures and dry conditions and is adapted to arid and semi-arid climates. Optimum temperature for root development is 77 to 95° F. When moisture is limited, the plant stops growing but does not die. While intermittent growth helps the plant survive drought, it also delays maturity. Growing season ranges from 60-90 days (determinate varieties) to 120-150 days (indeterminate varieties). Guar responds to irrigation during dry periods. It is grown without irrigation in areas with 10 to 40 in. of annual rainfall. Excessive rain or humidity after maturity causes the beans to turn black and shrivel, reducing their quality and marketability. While profitable seed production in southern U.S. areas of high rainfall and humidity is likely to be limited, guar can be successfully grown as a green manure crop under these conditions. Guar varieties that require a particular daylength to flower or day-neutral have been described. Considering these high soil temperatures and long growing season, successful guar production in Wisconsin and Minnesota is very unlikely.

Guar grows well under a wide range of soil conditions. It performs best on fertile, medium-textured and sandy loam soils with good structure and well-drained subsoils. Guar is susceptible to waterlogging. Guar is considered to be tolerant of both soil salinity and alkalinity. Guar is an excellent soil-improving crop and fits well in a crop-rotation program with grain sorghum, small grains or vegetables. In Australia, guar was found to add 1961b N/acre to the soil-plant system over three years. Increased yields can be expected from crops following guar because of increased soil nitrogen reserves. When used in rotation with cotton in Texas, researchers measured a 15% yield increase in cotton.

B. Seed Preparation, Germination, and Cultural Practices

Select seed that is uniform in color and size and is free from other crop and weed seed. New guar varieties have been released that have some resistance to diseases that once devastated fields of the crop. To prevent disease problems, select certified seed that does not contain seed of older varieties with less disease resistance. Seed should be inoculated just before planting with a special guar inoculant or the cowpea (Group “E”) inoculant. Exposure of inoculum to sunlight, heat and drying before planting can impair the effectiveness of the nitrogen-fixing bacteria. Seed should be planted in moist soil within 2 hours after inoculation. Fungicidal seed treatments may inhibit inoculation.

The seedbed should be firm and weed-free. Soil in the row should be ridged slightly to facilitate harvest of low-set beans. Guar should be planted when soil temperature is above 70° F.; the optimum soil temperature for germination is 86° F. A warm seedbed, adequate soil moisture and warm growing weather are essential for establishment of a stand. In Texas, June plantings of guar produce more reproductive buds than July plantings, resulting in substantially higher yields. Thus, production of this crop in the Upper Midwest is unlikely.

Guar is usually planted in 36 to 40 in. rows with a row crop planter. However, it can be broadcast seeded or planted in narrower rows with a grain drill if moisture is adequate. A planting depth of 1 to 1½ in. is usually recommended. If guar seed is crushed, gumming or clogging of equipment may occur. To prevent clogging, holes on the bottom sides of the plates should be straight, rather than beveled or tapered. Adding graphite or a dry detergent to the seed box and reducing seed weight on the plates by filling the planter box only about one-third full may also help prevent gumming during planting.

Although some studies have found little effect on yield when seeding rates ranged from 5 to 44 lb seed/acre, other researchers have indicated an optimum seeding rate of 5 to 9 viable seeds/ft of row (30 in. rows). Current Texas recommendations are 5 lb/acre for 30 in. rows and 12 lb/acre for broadcast. Broadcasting should be practiced only where moisture is sufficient to support the higher plant population.

Nitrogen is not thought to be limiting in guar when the plants are well modulated. Like most legumes, guar usually requires application of a rather high level of phosphorus (20 to 30 lb of P₂O₅/acre) and medium levels of potash (40 to 50 lb of K₂O/acre). For highest yield, fertilize according to soil test results. Apply fertilizer below the seed before planting or to the side and below the seed at planting. Sulfur fertilizers have been found to affect guar on some soils, and zinc deficiency is a common problem in India. Moderately alkaline soils are considered desirable for guar crop production (pH 7.0 to 8.0).

C. Variety Selection

There have been notable improvements in guar varieties developed in the last 30 years. The newer cultivars are much more disease resistant with higher yields. Pod set in improved varieties is higher, and pods are well distributed on the main stem and branches, increasing harvest efficiency. The multiple branching of these newer cultivars also produces more pods. Only the earliest-maturing varieties are recommended for production in Wisconsin and Minnesota.

Brooks, released in 1964, was the first improved variety, replacing Texsel and Groehler. Brooks has been grown on most of the guar acreage since 1966, but is rapidly being replaced by two newer releases, Kinman and Esser. Brooks is high-yielding and resistant to the major guar diseases, Alternaria leaf spot and bacterial blight. It is medium to late in maturity. Plants have a fine-branching growth habit and small racemes of medium-sized pods. Leaves and stem are glabrous. The seed is of medium size.

Hall is a slightly later-maturing variety than Brooks, and therefore not recommended for production in the Upper Midwest. It is resistant to bacterial blight and Alternaria leaf spot. Plants are relatively tall, coarse, finely branched, and produce small racemes of medium-sized pods. Leaves and stems are glabrous. This variety is best adapted to heavier soil types and higher elevations.

Mills is an early-maturing variety which also is resistant to bacterial blight and Alternaria leaf spot. Plants are short and finely branched and produce small racemes with relatively large pods. Leaves and stems are pubescent (hairy). Seeds are larger than those of Brooks and Hall. In dry seasons, Mills does not grow tall enough for efficient harvest. Yields are generally lower than those of Brooks and Hall.

Kinman, released by the Texas Agricultural Experiment Station, the USDA-ARS and the Oklahoma Agricultural Experiment Station in 1975, is derived from Brooks and Mills. Kinman is about 7 days earlier in maturity than Hall and of the same maturity as Brooks. It is highly resistant to bacterial leaf blight and Alternaria leaf spot. Kinman is slightly taller and coarser-stemmed than Brooks, but less so than Hall. It is fine branched and produces small-to-medium sized racemes. Seed pods are medium in length and generally contain from 7 to 9 seeds each. Seed of Kinman is slightly larger than Brooks. In 41 yield trials at eight locations in Texas and Oklahoma from 1971 to 1976, Kinman produced 17% more seed than Brooks.

Esser, released with Kinman in 1975, is a selection from progeny of the same Brooks×Mills cross. It is medium to late in maturity and therefore is probably not a good cultivar for Wisconsin and Minnesota. It has high resistance to Alternaria leaf spot and bacterial leaf blight. Esser has shown superior disease tolerance to Brooks and Kinman under severe bacterial blight conditions. Esser plants have Brooks' fine branching growth habit, but Esser has stronger main stems and fewer lateral branches. Esser produces small racemes with medium-sized pods.

Lewis, released by the Texas Agricultural Station and the USDA-ARS in 1986, is a selection from a cross of a glabrous parent with a pubescent (hairy) parent. Lewis is a medium-to-late maturing variety that is highly resistant to Alternaria leaf spot and bacterial leaf blight. Leaves, stems and pods are glabrous. Plants have a basal branching growth habit. The main stem and the basal branches possess short internodes with racemes initiated at each node over the entire plant. Plants are of average height, and racemes and pods are of medium length. Pods generally contain 5 to 9 seeds of average size. In 10 yield tests at five Texas locations during 1980-1983, Lewis produced mean seed yields approximately 25% higher than Kinman and 21% higher than Esser.

D. Weed and Disease Control

Young guar plants grow slowly and are particularly susceptible to weed problems. Weeds can reduce yields and create harvesting problems. Guar should not be seeded in fields heavily infested with Johnsongrass (Sorghum halepense) and other perennial weeds. Early preparation of land and mechanical cultivations during the growing season will help minimize weed problems. Covering the lower branches during cultivation may promote development of disease and increase harvest difficulties.

Treflan (trifluralin) is registered for use on guar as a preplant incorporated treatment to control most annual grass and several annual broadleaf weeds. Follow label instructions carefully for different soil types.

Selecting disease-resistant varieties and high-quality certified seed is the best defense against disease problems. There are two major diseases of guar worldwide: alternaria leaf or target spot (Alternaria cucumerina var. cyamopsidis; this fungal disease may become severe during periods of heavy dew and high humidity; causes a brown target-like lesion on the leaf between bloom and pod set; as the disease progresses, lesions enlarge, join and cause leaf drop); and bacterial blight (Xanthomonas cyamopsidis; this seed-borne disease can cause loss of plants from the seedling stage until maturity; symptoms include large angular necrotic lesions at the tips of leaves, which cause defoliation and black streaking of the stems; potentially the greatest disease hazard to guar).

The guar midge (Contarinia texana) is the primary guar insect pest in the Southwest. Heavy midge infestations have caused up to 30% loss in seed production. Guar midge infestations are generally heavier in fields with sandy or sandy loam soils.

Damage to guar is caused by the larvae, which develop in the guar buds. Infested buds eventually dry up and fall from the plant. The adult female midge deposits her eggs in developing buds. After larvae complete their development, they drop from the buds to the ground to pupate. There are several generations each year.

Rainfall or sprinkler irrigation can reduce midge populations drastically. However, field inspection should continue because midge infestation problems may increase again as a result of improved growing conditions. Control midges while guar is producing buds—primarily between 45 and 90 days after emergence.

Other guar insect pests include the gall midge (Asphondylia sp.), three-cornered alfalfa hoppers, pea aphids, white grubs, thrips, and whiteflies. Storage pests have not been a problem with guar.

E. Harvesting, Drying and Storage

Since guar beans generally do not shatter, the crop can be direct-combined as soon after maturity as possible. Harvest does not generally take place until after frost in northern regions. At maturity, the seed pods are brown and dry, and seed moisture content is less than 14%. GRAMOXONE (paraquat) can be used as a harvest aid to speed up drying and to kill weeds prior to frost. Apply when pods are fully mature. Preharvest interval is 4 days. Do not graze treated areas or use the treated forage for animal feed.

Guar beans can be harvested with an ordinary grain combine. The cylinder should be slowed and the combine speed reduced to a rate that will permit proper threshing of the beans. A high fan speed can be used to clean out foreign material. Reel speed should be slightly greater than combine ground speed. Improper reel speed can shatter seed pods. Reels should be set just deep enough in the guar to control the stalks, and should be about 6 to 12 in. ahead of the cutterbar. Some operators replace the wooden reel bats with ½ in. steel rods to reduce shattering. When harvested for hay, guar leaves drop readily unless extreme care is taken during the curing process. For hay, the crop should be cut when the first lower pods turn brown.

Guar can be harvested for seed and then plowed under or used as a mulch. If seed is not harvested, guar used for green manure should be turned under when the lower pods begin to turn brown. Following harvest, the seed is graded for size and cleaned to remove shrunken seed and crop residue. Little information is available on optimum storage conditions for guar, but this has not been identified as a problem in most production guides. Following cleaning, milling for gum extraction may proceed. The principal factors that decrease seed quality in guar are seed blackening and the production of small and shrunken seed. White seed is preferred for many food applications, and black seed is often discounted. Darkening tends to follow patterns of increasing rainfall, especially when it occurs during the period of seed maturation. Small seed contains less endosperm and therefore is less desirable for milling. Late flowering, diseases, insects and low moisture can cause small seed (preferred size is 4 mm).

F. Yield Potential, Performance Results and Economics

Production practices and rainfall during the growing season cause seed yields to vary from about 300 to 2,000 lb/acre. Yields of several varieties in Texas were reported to average about 700-1100 lb/a (Undersander et al., 1991). Experimental plantings of guar at Rosemont, Minnesota, have resulted in plants that bloomed but produced very little seed. Income and production costs for guar vary from year to year and according to soil types. Production costs often vary by $20 to $40/acre between farms because of different fertilizer usage and other production practices.

Demand for guar is increasing because of the wide use of the gum in more products and efforts of dealers to obtain a larger percentage of the gum from domestic sources. Growth in the early 1980s was estimated at 10% annually. Grade factors considered by the purchaser are the moisture, foreign material and test weight. Identify a market and secure a contract, if possible, before growing guar for bean production. The value of guar as a soil builder to increase yields of succeeding crops should not be overlooked when considering guar as an alternative crop.

G. Information Sources

The following papers provide additional information on guar biology: Jackson and Doughton; Kinman and Esser, 1975; Lewis, 1986; Tripp et al., Whistler and Hymowitz, 1979 (each of the preceding is incorporated by reference in its entirety).

### II. AGROBACTERIUM

A. Transformation

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells generally is well known in the art. See, for example, the methods described by Fraley et al. (1985), Rogers et al. (1987) and U.S. Pat. No. 5,563,055, specifically incorporated herein by reference in its entirety. Of particular interest in transformation protocols is A. tumefaciens.

It is understood that by Agrobacterium, applicants include Rhizobia species known to act in the same manner as, for example, A. tumefaciens for purposes of plant transformation. Such species include Rhizobium spp., including Rhizobium leguminosarum and the like.

Agrobacterium-mediated transformation is most efficient in dicotyledonous plants and is the preferable method for transformation of dicots, including Arabidopsis, tobacco, tomato, alfalfa and potato. Indeed, while Agrobacterium-mediated transformation has been routinely used with dicotyledonous plants for a number of years, it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al., 1997; U.S. Pat. No. 5,591,616, specifically incorporated herein by reference in its entirety), wheat (McCormac et al., 1998), barley (Tingay et al., 1997; McCormac et al., 1998), alfalfa (Thomas et al., 1990) and maize (Ishida et al., 1996).

Modern Agrobacterium transformation vectors are capable of replication in E. coli as well as Agrobacterium, allowing for convenient manipulations as described (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described (Rogers et al., 1987) have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant strains where Agrobacterium-mediated transformation is efficient, it is the method of choice because of the facile and defined nature of the gene transfer.

Tissue cultures may be used in certain transformation techniques for the preparation of cells for transformation and for the regeneration of plants therefrom. Maintenance of tissue cultures requires use of media and controlled environments. “Media” refers to the numerous nutrient mixtures that are used to grow cells in vitro, that is, outside of the intact living organism. The medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. Rate of cell growth also will vary among cultures initiated with the array of media that permit growth of that cell type.

Tissue that can be grown in a culture includes meristem cells, Type I, Type II, and Type III callus, immature embryos and gametic cells such as microspores, pollen, sperm and egg cells. Type I, Type II, and Type III callus may be initiated from tissue sources including, but not limited to, immature embryos, seedling apical meristems, root, leaf, microspores and the like. Those cells which are capable of proliferating as callus also are recipient cells for genetic transformation.

Somatic cells are of various types. Embryogenic cells are one example of somatic cells which may be induced to regenerate a plant through embryo formation. Non-embryogenic cells are those which typically will not respond in such a fashion. Certain techniques may be used that enrich recipient cells within a cell population. For example, Type II callus development, followed by manual selection and culture of friable, embryogenic tissue, generally results in an enrichment of cells. Manual selection techniques which can be employed to select target cells may include, e.g., assessing cell morphology and differentiation, or may use various physical or biological means. Cryopreservation also is a possible method of selecting for recipient cells.

Where employed, cultured cells may be grown either on solid supports or in the form of liquid suspensions. In either instance, nutrients may be provided to the cells in the form of media, and environmental conditions controlled. There are many types of tissue culture media comprised of various amino acids, salts, sugars, growth regulators and vitamins. Most of the media employed in the practice of the invention will have some similar components, but may differ in the composition and proportions of their ingredients depending on the particular application envisioned. For example, various cell types usually grow in more than one type of media, but will exhibit different growth rates and different morphologies, depending on the growth media. In some media, cells survive but do not divide. Various types of media suitable for culture of plant cells previously have been described. Examples of these media include, but are not limited to, the N6 medium described by Chu et al. (1975) and MS media (Murashige and Skoog, 1962).

The following patents relate to Agrobacterium transformation methods and are hereby incorporated by reference: U.S. Pat. Nos. 6,846,971, 6,822,144, 6,800,791, 6,759,573, 6,696,622. 6,686,515, 6,664,108, 6,620,986, 6,603,061, 6,455,761, 6,420,630, 6,384,301, 6,369,298, 6,323,396, 6,307,127, 6,300,545, 6,274,791, 6,265,638, 6,255,559, 6,255,115, 6,215,051, 6,162,965, 6,103,955, 6,074,877, 6,074,876, 6,051,757, 6,040,498, 6,037,522, 5,994,624, 5,981,840, 5,977,439, 5,952,543, 5,948,956, 5,932,782, 5,929,300, 5,922,928, 5,919,919, 5,846,797, 5,824,877, 5,824,872, 5,750,871, 5,733,744, 5,712,112, 5,693,512, 5,689,053, 5,591,616, 5,589,615, 5,569,834, 5,565,347, 5,563,055, 5,530,182, 5,463,174, 5,416,011, 5,262,316, 5,188,958, 5,159,135, 5,004,863, 4,954,442, and 4,795,855.

B. Agrobacterium rhizogenes

Agrobacterium rhizogenes is a Gram negative soil bacterium that can be utilized for gene transfer in plants. Wounding of plants in the rhizosphere, or zone that surrounds the roots, leads to the secretion of phenolics which effects that attract the bacteria. Under such conditions, certain bacterial genes are turned on leading to the transfer of its T-DNA from its Ri plasmid into the plant through the wound. After integration and expression, a “hairy root” phenotype is observed. The hairy roots are useful where stimulation of root biomass is desired. More information on A. rhizogenes can be found in Otani et al. (1993), Van de Velde et al. (2003), and Intrieri and Buiatti (2001).

### III. TRANSFORMATION CONSTRUCTS, NUCLEIC ACIDS AND POLYPEPTIDES

Various coding sequences may be provided operably linked to a heterologous promoter, in either sense or antisense orientation and used to transform guar plants according to the present invention. Agrobacterium expression constructs may be constructed, comprising such sequences, as may be plants and plant cells transformed with the sequences. The construction of Agrobacterium vectors which may be employed in conjunction with plant transformation techniques using these or other sequences according to the invention will be known to those of skill of the art in light of the present disclosure. The techniques of the current invention are thus not limited to any particular nucleic acid sequences, and the following merely provide examples of genes suitable for transfer and expression into plants.

A. Genes

i. Lignin Biosynthesis

One example of a beneficial modification that may be made to plants is to lignin content. Lignin is a major structural component of secondarily thickened plant cell walls. It is a complex polymer of hydroxylated and methoxylated phenylpropane units, linked via oxidative coupling (Boudet et al., 1995). Because of the negative effects of lignin on forage quality, there is considerable interest in genetic manipulation to alter the quantity and/or quality of the lignin polymer (Dixon et al., 1996). At the same time, lignin is important for stem rigidity and hydrophobicity of vascular elements, and, particularly in cereal crops, may be an important inducible defensive barrier against fungal pathogen attack (Beardmore et al., 1983). Thus, lignin modification must not compromise basic functions for the plant and thereby result in negative traits such as lodging or disease susceptibility.

Examples of genes that may be modified include enzymes of the monolignol pathway, such as caffeic acid 3-O-methyltransferase (COMT), caffeoyl CoA 3-O-methyltransferase (CCoAOMT) and cinnamyl alcohol dehydrogenase (CAD). Constitutive cauliflower mosaic virus 35S promoter-driven antisense reduction of COMT to less than 5% of wild-type values in the tropical pasture legume Stylosanthes humilis resulted in a strong reduction in S lignin based on histochemical analysis, for example (Rae et al., 2001). In vitro digestibility of stem material in rumen fluid was increased by up to 10% in the transgenic plants exhibiting strongest COMT down-regulation.

ii. Herbicide Resistance

Numerous herbicide resistance genes are known and may be employed with the invention. An example is a gene conferring resistance to a herbicide that inhibits the growing point or meristem, such as an imidazalinone or a sulfonylurea. Exemplary genes in this category code for mutant ALS and AHAS enzyme as described, for example, by Lee et al. (1988); Gleen et al. (1992) and Miki et al. (1990).

Resistance genes for glyphosate (resistance conferred by mutant 5-enolpyruvl-3 phosphikimate synthase (EPSP) and aroA genes, respectively) and other phosphono compounds such as glufosinate (phosphinothricin acetyl transferase (PAT) and Streptomyces hygroscopicus phosphinothricin-acetyl transferase (bar) genes) may also be used. See, for example, U.S. Pat. No. 4,940,835, which discloses the nucleotide sequence of a form of EPSPS which can confer glyphosate resistance. Examples of specific EPSPS transformation events conferring glyphosate resistance are provided by U.S. Pat. No. 6,040,497.

A DNA molecule encoding a mutant aroA gene can be obtained under ATCC accession number 39256, and the nucleotide sequence of the mutant gene is disclosed in U.S. Pat. No. 4,769,061 to Comai. EPA No. 0 333 033 and U.S. Pat. No. 4,975,374 disclose nucleotide sequences of glutamine synthetase genes which confer resistance to herbicides such as L-phosphinothricin. The nucleotide sequence of a phosphinothricin-acetyltransferase gene is provided in EPA No. 0 242 246. DeGreef et al. (1989), describe the production of transgenic plants that express chimeric bar genes coding for phosphinothricin acetyl transferase activity. Exemplary of genes conferring resistance to phenoxy propionic acids and cycloshexones, such as sethoxydim and haloxyfop are the Acct-S1, Accl-S2 and Acct-S3 genes described by Marshall et al. (1992).

Genes are also known conferring resistance to a herbicide that inhibits photosynthesis, such as a triazine (psbA and gs+ genes) and a benzonitrile (nitrilase gene). Przibila et al. (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441, and 67442. Cloning and expression of DNA coding for a glutathione S-transferase is described by Hayes et al. (1992).

iii. Disease Resistance

Plant defenses are often activated by specific interaction between the product of a disease resistance gene (R) in the plant and the product of a corresponding avirulence (Avr) gene in the pathogen. A plant line can be transformed with cloned resistance gene to engineer plants that are resistant to specific pathogen strains. See, for example Jones et al. (1994) (cloning of the tomato Cf-9 gene for resistance to Cladosporium fulvum); Martin et al. (1993) (tomato Pto gene for resistance to Pseudomonas syringae pv.); and Mindrinos et al. (1994) (Arabidopsis RSP2 gene for resistance to Pseudomonas syringae).

A viral-invasive protein or a complex toxin derived therefrom may also be used for viral disease resistance. For example, the accumulation of viral coat proteins in transformed plant cells imparts resistance to viral infection and/or disease development effected by the virus from which the coat protein gene is derived, as well as by related viruses. See Beachy et al. (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id. A virus-specific antibody may also be used. See, for example, Tavladoraki et al. (1993), who show that transgenic plants expressing recombinant antibody genes are protected from virus attack. Logemann et al. (1992), for example, disclose transgenic plants expressing a barley ribosome-inactivating gene have an increased resistance to fungal disease.

iv. Insect Resistance

One example of an insect resistance gene includes a Bacillus thuringiensis protein, a derivative thereof or a synthetic polypeptide modeled thereon. See, for example, Geiser et al. (1986), who disclose the cloning and nucleotide sequence of a Bt δ-endotoxin gene. Moreover, DNA molecules encoding δ-endotoxin genes can be purchased from the American Type Culture Collection, Manassas, Va., for example, under ATCC Accession Nos. 40098, 67136, 31995 and 31998. Another example is a lectin. See, for example, Van Damme et al. (1994), who disclose the nucleotide sequences of several Clivia miniata mannose-binding lectin genes. A vitamin-binding protein may also be used, such as avidin. See PCT application US93/06487, the contents of which are hereby incorporated by reference. This application teaches the use of avidin and avidin homologues as larvicides against insect pests.

Yet another insect resistance gene is an enzyme inhibitor, for example, a protease or proteinase inhibitor or an amylase inhibitor. See, for example, Abe et al. (1987) (nucleotide sequence of rice cysteine proteinase inhibitor), Huub et al. (1993) (nucleotide sequence of cDNA encoding tobacco proteinase inhibitor I), and Sumitani et al. (1993) (nucleotide sequence of Streptomyces nitrosporeus α-amylase inhibitor). An insect-specific hormone or pheromone may also be used. See, for example, the disclosure by Hammock et al. (1990), of baculovirus expression of cloned juvenile hormone esterase, an inactivator of juvenile hormone.

Still other examples include an insect-specific antibody or an immunotoxin derived therefrom and a developmental-arrestive protein. See Taylor et al. (1994), who described enzymatic inactivation in transgenic tobacco via production of single-chain antibody fragments.

v. Modified Fatty Acid, Phytate and Carbohydrate Metabolism

Genes may be used conferring modified fatty acid metabolism. For example, stearyl-ACP desaturase genes may be used. See Knutzon et al. (1992). Various fatty acid desaturases have also been described, such as a Saccharomyces cerevisiae OLE1 gene encoding A9-fatty acid desaturase, an enzyme which forms the monounsaturated palmitoleic (16:1) and oleic (18:1) fatty acids from palmitoyl (16:0) or stearoyl (18:0) CoA (McDonough et al., 1992); a gene encoding a stearoyl-acyl carrier protein delta-9 desaturase from castor (Fox et al., 1993); Δ6- and Δ12-desaturases from the cyanobacteria Synechocystis responsible for the conversion of linoleic acid (18:2) to gamma-linolenic acid (18:3 gamma) (Reddy et al., 1993); a gene from Arabidopsis thaliana that encodes an omega-3 desaturase (Arondel et al., 1992); plant Δ9-desaturases (PCT Application Publ. No. WO 91/13972) and soybean and Brassica Δ15 desaturases (European Patent Application Publ. No. EP 0616644).

Phytate metabolism may also be modified by introduction of a phytase-encoding gene to enhance breakdown of phytate, adding more free phosphate to the transformed plant. For example, see Van Hartingsveldt et al. (1993), for a disclosure of the nucleotide sequence of an Aspergillus niger phytase gene. In corn, this, for example, could be accomplished by cloning and then reintroducing DNA associated with the single allele which is responsible for corn mutants characterized by low levels of phytic acid. See Raboy et al. (2000).

A number of genes are known that may be used to alter carbohydrate metabolism. For example, plants may be transformed with a gene coding for an enzyme that alters the branching pattern of starch. See Shiroza et al. (1988) (nucleotide sequence of Streptococcus mutants fructosyltransferase gene), Steinmetz et al. (1985) (nucleotide sequence of Bacillus subtilis levansucrase gene), Pen et al. (1992) (production of transgenic plants that express Bacillus licheniformis α-amylase), Elliot et al. (1993) (nucleotide sequences of tomato invertase genes), Sergaard et al. (1993) (site-directed mutagenesis of barley α-amylase gene), and Fisher et al. (1993) (maize endosperm starch branching enzyme II). The Z10 gene encoding a 10 kD zein storage protein from maize may also be used to alter the quantities of 10 kD Zein in the cells relative to other components (Kirihara et al., 1988).

Of particular interest in guar transformation are genes affecting gum quality. The gum is comprised of a high molecular weight galactomannan sugar based on an β,1-4-linked mannan backbone. The sequence for the only published β-mannan synthase sequence can be found at GenBank Accession No. AY372247 (GI: 38532105). Three α-galactosyltransferases that catalyze the addition of the single unit α-1-6-linked galactose side chains can be found at GenBank Accession Nos. AJ938067 (GI: 62700754), AJ864710 (GI: 55956979) and AJ245478 (GI: 5702017).

B. Regulatory Elements

Exemplary promoters for expression of a nucleic acid sequence include plant promoters such as the CaMV 35S promoter (Odell et al., 1985), or others such as CaMV 19S (Lawton et al., 1987), nos (Ebert et al., 1987), Adh (Llewellyn et al., 1987), sucrose synthase (Yang and Russell, 1990), α-tubulin, actin (Wang et al., 1992), cab (Sullivan et al., 1989), PEPCase (Hudspeth and Grula, 1989) or those promoters associated with the R gene complex (Chandler et al., 1989). Tissue specific promoters such as root cell promoters (Conkling et al., 1990) and tissue specific enhancers (Fromm et al., 1986) are also contemplated to be useful, as are inducible promoters such as ABA- and turgor-inducible promoters. In one embodiment of the invention, the native promoter of an acid phosphatase coding sequence is used.

The DNA sequence between the transcription initiation site and the start of the coding sequence, i.e., the untranslated leader sequence, can also influence gene expression. One may thus wish to employ a particular leader sequence with a transformation construct of the invention. Preferred leader sequences are contemplated to include those which comprise sequences predicted to direct optimum expression of the attached gene, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants will typically be preferred.

Vectors for use in tissue-specific targeting of genes in transgenic plants will typically include tissue-specific promoters and may also include other tissue-specific control elements such as enhancer sequences. Promoters which direct specific or enhanced expression in certain plant tissues will be known to those of skill in the art in light of the present disclosure. These include, for example, the rbcS promoter, specific for green tissue; the ocs, nos and mas promoters which have higher activity in roots.

C. Terminators

Transformation constructs prepared in accordance with the invention will typically include a 3′ end DNA sequence that acts as a signal to terminate transcription and allow for the poly-adenylation of the mRNA produced by coding sequences operably linked to a promoter. In one embodiment of the invention, the native terminator of an acid phosphatase coding sequence is used. Alternatively, a heterologous 3′ end may enhance the expression of sense or antisense acid phosphatase coding sequences. Examples of terminators that are deemed to be useful in this context include those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II genes from potato or tomato. Regulatory elements such as an Adh intron (Callis et al., 1987), sucrose synthase intron (Vasil et al., 1989) or TMV omega element (Gallie et al., 1989), may further be included where desired.

D. Transit or Signal Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

E. Marker Genes

By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Of course, many examples of suitable marker proteins are known to the art and can be employed in the practice of the invention.

Included within the terms selectable or screenable markers also are genes which encode a “secretable marker” whose secretion can be detected as a means of identifying or selecting for transformed cells. Examples include markers which are secretable antigens that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR-S).

Many selectable marker coding regions are known and could be used with the present invention including, but not limited to, neo (Potrykus et al., 1985), which provides kanamycin resistance and can be selected for using kanamycin, G418, paromomycin, etc.; bar, which confers bialaphos or phosphinothricin resistance; a mutant EPSP synthase protein (Hinchee et al., 1988) conferring glyphosate resistance; a nitrilase such as bxn from Klebsiella ozaenae which confers resistance to bromoxynil (Stalker et al., 1988); a mutant acetolactate synthase (ALS) which confers resistance to imidazolinone, sulfonylurea or other ALS inhibiting chemicals (European Patent Application 154 204, 1985); a methotrexate resistant DHFR (Thillet et al., 1988), a dalapon dehalogenase that confers resistance to the herbicide dalapon; or a mutated anthranilate synthase that confers resistance to 5-methyl tryptophan.

An illustrative embodiment of selectable marker capable of being used in systems to select transformants are those that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, (Murakami et al., 1986; Twell et al., 1989) causing rapid accumulation of ammonia and cell death.

Screenable markers that may be employed include a β-glucuronidase (GUS) or uidA gene which encodes an enzyme for which various chromogenic substrates are known; an R-locus gene, which encodes a product that regulates the production of anthocyanin pigments (red color) in plant tissues (Dellaporta et al., 1988); a β-lactamase gene (Sutcliffe, 1978), which encodes an enzyme for which various chromogenic substrates are known (e.g., PADAC, a chromogenic cephalosporin); a xylE gene (Zukowsky et al., 1983) which encodes a catechol dioxygenase that can convert chromogenic catechols; an α-amylase gene (Ikuta et al., 1990); a tyrosinase gene (Katz et al., 1983) which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene (Ow et al., 1986), which allows for bioluminescence detection; an aequorin gene (Prasher et al., 1985) which may be employed in calcium-sensitive bioluminescence detection; or a gene encoding for green fluorescent protein (Sheen et al., 1995; Haseloff et al., 1997; Reichel et al., 1996; Tian et al., 1997; WO 97/41228).

Another screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It also is envisioned that this system may be developed for populational screening for bioluminescence, such as on tissue culture plates, or even for whole plant screening. The gene which encodes green fluorescent protein (GFP) is also contemplated as a particularly useful reporter gene (Sheen et al., 1995; Haseloff et al., 1997; Reichel et al., 1996; Tian et al., 1997; WO 97/41228). Expression of green fluorescent protein may be visualized in a cell or plant as fluorescence following illumination by particular wavelengths of light.

### IV. SELECTION, PRODUCTION AND CHARACTERIZATION OF STABLY TRANSFORMED GUAR PLANTS

After effecting delivery of exogenous DNA to recipient guar cells, steps generally concern identifying the transformed cells for further culturing and plant regeneration. In order to improve the ability to identify transformants, one may desire to employ a selectable or screenable marker gene with a transformation vector prepared in accordance with the invention. In this case, one would then generally assay the potentially transformed cell population for example, a stolon, by exposing the cells to a selective agent or agents, or one would screen the cells for the desired marker gene trait.

A. Selection

It is believed that DNA is introduced into only a small percentage of target cells in any one study. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphostransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

One herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism (Ogawa et al., 1973). Synthetic PPT, the active ingredient in the herbicide Liberty™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity (Thompson et al., 1987). The bar gene has been cloned (Murakami et al., 1986; Thompson et al., 1987) and expressed in transgenic tobacco, tomato, potato (De Block et al., 1987), Brassica (De Block et al., 1989) and maize (U.S. Pat. No. 5,550,318). In previous reports, some transgenic plants which expressed the resistance gene were completely resistant to commercial formulations of PPT and bialaphos in greenhouses.

Another example of a herbicide which is useful for selection of transformed cell lines in the practice of the invention is the broad spectrum herbicide glyphosate. Glyphosate inhibits the action of the enzyme EPSPS which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, International Patent WO 97/4103. The best characterized mutant EPSPS gene conferring glyphosate resistance comprises amino acid changes at residues 102 and 106, although it is anticipated that other mutations will also be useful (PCT/WO97/4103).

To use the bar-bialaphos or the EPSPS-glyphosate selective system, transformed tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate will typically be preferred, it is proposed that ranges of 0.1-50 mg/l bialaphos or 0.1-50 mM glyphosate will find utility.

An example of a screenable marker trait is the enzyme luciferase. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. These assays are nondestructive and transformed cells may be cultured further following identification. The photon counting camera is especially valuable as it allows one to identify specific cells or groups of cells which are expressing luciferase and manipulate those in real time. Another screenable marker which may be used in a similar fashion is the gene coding for green fluorescent protein.

It further is contemplated that combinations of screenable and selectable markers will be useful for identification of transformed cells. In some cell or tissue types a selection agent, such as bialaphos or glyphosate, may either not provide enough killing activity to clearly recognize transformed cells or may cause substantial nonselective inhibition of transformants and nontransformants alike, thus causing the selection technique to not be effective. It is proposed that selection with a growth inhibiting compound, such as bialaphos or glyphosate at concentrations below those that cause 100% inhibition followed by screening of growing tissue for expression of a screenable marker gene such as luciferase would allow one to recover transformants from cell or tissue types that are not amenable to selection alone. It is proposed that combinations of selection and screening may enable one to identify transformants in a wider variety of cell and tissue types. This may be efficiently achieved using a gene fusion between a selectable marker gene and a screenable marker gene, for example, between an NPTII gene and a GFP gene.

B. Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. One such growth regulator is dicamba or 2,4-D. However, other growth regulators may be employed, including NAA, NAA+2,4-D or picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 wk, then transferred to media conducive to maturation of embryoids. Cultures are transferred every 2 wk on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soiless plant growth mix, and hardened, e.g., in an environmentally controlled chamber, for example, at about 85% relative humidity, 600 ppm CO₂, and 25-250 microeinsteins m⁻² s⁻¹ of light. Plants may be matured in a growth chamber or greenhouse. Plants can be regenerated from about 6 wk to 10 months after a transformant is identified, depending on the initial tissue. During regeneration, cells are grown on solid media in tissue culture vessels. Illustrative embodiments of such vessels are petri dishes and PLANTCONs (MP Biomedical, Solon, Ohio). Regenerating plants can be grown at about 19 to 28° C. After the regenerating plants have reached the stage of shoot and root development, they may be transferred to a greenhouse for further growth and testing.

Seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 μl agarose. In embryo rescue, large embryos (defined as greater than 3 mm in length) are germinated directly on an appropriate media. Embryos smaller than that may be cultured for 1 wk on media containing the above ingredients along with 10⁻⁵M abscisic acid and then transferred to growth regulator-free medium for germination.

C. Characterization

To confirm the presence of the exogenous DNA or “transgene(s)” in the regenerating plants, a variety of assays may be performed. Such assays include, for example, “molecular biological” assays, such as Southern and Northern blotting and PCR™; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

D. DNA Integration, RNA Expression and Inheritance

Genomic DNA may be isolated from cell lines or any plant parts to determine the presence of the exogenous gene through the use of techniques well known to those skilled in the art. Note, that intact sequences will not always be present, presumably due to rearrangement or deletion of sequences in the cell. The presence of DNA elements introduced through the methods of this invention may be determined, for example, by polymerase chain reaction (PCR™). Using this technique, discreet fragments of DNA are amplified and detected by gel electrophoresis. This type of analysis permits one to determine whether a gene is present in a stable transformant, but does not prove integration of the introduced gene into the host cell genome. It is typically the case, however, that DNA has been integrated into the genome of all transformants that demonstrate the presence of the gene through PCR™ analysis. In addition, it is not typically possible using PCR™ techniques to determine whether transformants have exogenous genes introduced into different sites in the genome, i.e., whether transformants are of independent origin. It is contemplated that using PCR™ techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™, e.g., the presence of a gene, but also demonstrates integration into the genome and characterizes each individual transformant.

It is contemplated that using the techniques of dot or slot blot hybridization which are modifications of Southern hybridization techniques one could obtain the same information that is derived from PCR™, e.g., the presence of a gene. Both PCR™ and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR™ techniques also may be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species.

E. Gene Expression

While Southern blotting and PCR™ may be used to detect the gene(s) in question, they do not provide information as to whether the corresponding protein is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and ¹⁴C-acetyl CoA or for anthranilate synthase activity by following loss of fluorescence of anthranilate, to name two.

Very frequently the expression of a gene product is determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

### V. BREEDING PLANTS OF THE INVENTION

In addition to direct transformation of a particular guar plant genotype with a construct according to the current invention, transgenic guar plants may be made in crosses with a transformed guar plant having a selected DNA and a second guar plant. Therefore, the current invention not only encompasses a plant directly transformed or regenerated from cells which have been transformed in accordance with the current invention, but also the progeny of such plants. As used herein the term “progeny” denotes the offspring of any generation of a parent plant prepared in accordance with the instant invention, wherein the progeny comprises a selected DNA construct prepared in accordance with the invention. “Crossing” a plant to provide a plant line having one or more added transgenes relative to a starting plant line, as disclosed herein, is defined as the techniques that result in a transgene of the invention being introduced into a plant line by crossing a starting line with a donor plant line that comprises a transgene of the invention. To achieve this one could, for example, perform the following steps:
(a) plant seeds of the first (starting line) and second (donor plant line that comprises a transgene) parent plants;
(b) grow the seeds of the first and second parent plants into plants that bear flowers;
(c) pollinate a flower from the first parent plant with pollen from the second parent plant; and
(d) harvest seeds produced on the parent plant bearing the fertilized flower.
   Backcrossing is herein defined as the process including the steps of:
(a) crossing a plant of a first genotype containing a desired gene, DNA sequence or element to a plant of a second genotype lacking the desired gene, DNA sequence or element;
(b) selecting one or more progeny plant containing the desired gene, DNA sequence or element;
(c) crossing the progeny plant to a plant of the second genotype; and
(d) repeating steps (b) and (c) for the purpose of transferring a desired DNA sequence from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

### VI. DEFINITIONS

Expression: The combination of intracellular processes, including transcription and translation undergone by a coding DNA molecule such as a structural gene to produce a polypeptide.

Genetic Transformation: A process of introducing a DNA sequence or construct (e.g., a vector or expression cassette) into a cell or protoplast in which that exogenous DNA is incorporated into a chromosome or is capable of autonomous replication.

Heterologous: A sequence which is not normally present in a given host genome in the genetic context in which the sequence is currently found In this respect, the sequence may be native to the host genome, but be rearranged with respect to other genetic sequences within the host sequence. For example, a regulatory sequence may be heterologous in that it is linked to a different coding sequence relative to the native regulatory sequence.

Obtaining: When used in conjunction with a transgenic plant cell or transgenic plant, obtaining means either transforming a non-transgenic plant cell or plant to create the transgenic plant cell or plant, or planting transgenic plant seed to produce the transgenic plant cell or plant. Such a transgenic plant seed may be from an R₀ transgenic plant or may be from a progeny of any generation thereof that inherits a given transgenic sequence from a starting transgenic parent plant.

Promoter: A recognition site on a DNA sequence or group of DNA sequences that provides an expression control element for a structural gene and to which RNA polymerase specifically binds and initiates RNA synthesis (transcription) of that gene.

R₀ transgenic plant: A plant that has been genetically transformed or has been regenerated from a plant cell or cells that have been genetically transformed.

Regeneration: The process of growing a plant from a plant cell (e.g., plant protoplast, callus or explant).

Selected DNA: A DNA segment which one desires to introduce or has introduced into a plant genome by genetic transformation.

Stolon: A stolon is a specialized type of horizontal above-ground shoot, a colonizing organ that arises from an axillary bud near the base of the plant. The stolon differs from the typical vegetative shoot of that same plant in having much longer and, typically, thinner internodes, and the horizontal stolon also has a strong tendency to form adventitious roots at the nodes.

Transformation construct: A chimeric DNA molecule which is designed for introduction into a host genome by genetic transformation. Preferred transformation constructs will comprise all of the genetic elements necessary to direct the expression of one or more exogenous genes. In particular embodiments of the instant invention, it may be desirable to introduce a transformation construct into a host cell in the form of an expression cassette.

Transformed cell: A cell the DNA complement of which has been altered by the introduction of an exogenous DNA molecule into that cell.

Transgene: A segment of DNA which has been incorporated into a host genome or is capable of autonomous replication in a host cell and is capable of causing the expression of one or more coding sequences. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

Transgenic plant: A plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences which are native to the plant being transformed, but wherein the “exogenous” gene has been altered in order to alter the level or pattern of expression of the gene, for example, by use of one or more heterologous regulatory or other elements.

Vector: A DNA molecule designed for transformation into a host cell. Some vectors may be capable of replication in a host cell. A plasmid is an exemplary vector, as are expression cassettes isolated therefrom.

### VII. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Materials and Methods

Explant preparation. Guar seeds of two American cultivars (Lewis and Kinman; e.g. (Undersander et al., 1991) were surface sterilized in concentrated sulphuric acid for 10 min followed by four washes in ice cold sterile distilled water. Subsequently the seeds were treated with 10% commercial bleach (containing 6.0% free chlorine) for 5 min with a few drops of Tween 20 and washed thoroughly three to four times in sterile distilled water. The sterile seeds were sown in magenta boxes on MS medium (Murashige and Skoog, 1962) with 3% sucrose, 0.8% agar, pH 5.8, and germination was continued for 7-9 days under a 16 h/8 h day/night regime at 25° C. Three-four mm long cotyledonary node explants comprising the meristematic zone were excised from the seedlings and used for shoot regeneration and transformation.

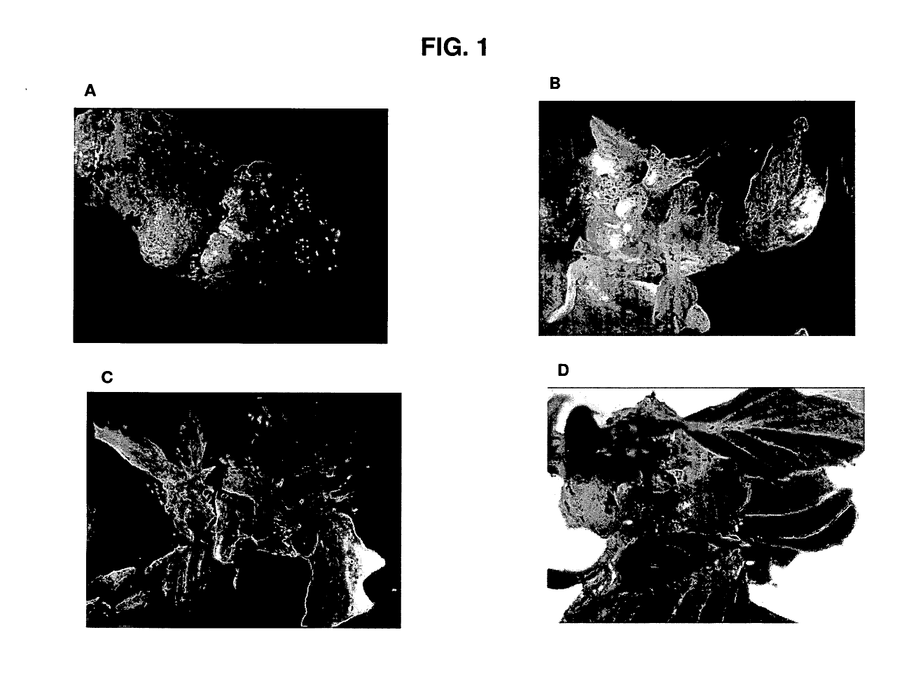

Shoot regeneration. Shoot regeneration was performed in two different phases; shoot initiation and shoot elongation. For shoot initiation, 5-10 mm cotyledonary node explants were cultured with the adaxial surface touching the medium in Petri plates (FIG. 1A). The explants were cultured on Gamborg's B5 medium (Gamborg et al., 1968), supplemented with 3% (w/v) sucrose, 0.8% Phyto agar (Caisson Laboratories Inc.), pH 5.8 (shoot initiation medium, SIM) with different combinations of growth regulators (Table 1). Cultures were incubated at 25° C. under a 16 h/8 h photoperiod for 10 days and subsequently transferred to the shoot elongation medium (SEM-MS salts, B5 vitamins, 0.1 mg/l indole 3-acetic acid (IAA), 0.5 mg/l gibberellic acid A3 (GA3), 3% sucrose, 3 mM morpholine ethane sulfonic acid (MES), 0.8% agar, pH 5.8).

Transformation and selection. Suspension cultures of five different Agrobacterium tumefaciens strains (LBA 4404, C58C1, AGL1, GV 3101 and EHA 105) harboring the plasmid pCambia 2301 (Cambia) containing a β-glucuronidase gene (uid A) and a neomycin phosphotransferase gene (nptII), both driven by cauliflower mosaic virus 35S promoters, were prepared as overnight cultures in YEP medium (yeast extract, 10 g/l; peptone, 10 g/l; sodium chloride, 5 g/l; pH 7.0) containing 50 mg/l kanamycin sulphate and 100 mg/l rifampicin. A 50 ml culture was centrifuged at 3000 rpm at 4° C. for 20 min. The pellet was suspended and diluted to OD 0.1-1.0 (at 660 nm) in liquid co-cultivation medium (B5 salts, B5 vitamins, 20 g/l sucrose, 1.0 mM silver thiosulphate, 100 mM acetosyringone, pH 5.7). Cotyledonary node explants were incubated in the Agrobacterium suspension in co-cultivation medium for 25 min, blotted on sterile filter paper and transferred to solid co-cultivation medium (liquid co-cultivation medium solidified with 0.8% agar). After 2-3 days co-cultivation at 25° C. and 16 h/8 h photoperiod the infected explants were washed twice (30 min each time) in liquid B5 medium with 100 mg/l carbenicillin, 100 mg/l cefotaxime and 1000 mg/l lysozyme, stirring at 80 rpm on a rotary shaker. After washing and blotting on sterile filter paper, the explants were transferred to selection medium (Gamborg's B5 medium, Thidiazuron 4.55 μM, 6-benzylaminopurine 4.44 mM, silver thiosulphate 1.0 mM, nickel chloride 1 mg/l, cefotaxime 250 mg/l, carbenicillin 250 mg/l, kanamycin sulphate 25-125 mg/l, 2% (w/v) sucrose, 0.8% Phyto agar (PhytoTechnology Laboratories, Shawnee Mission, Kans.), pH 5.8) and incubated at 25° C. under a 16 h/8 h day/night photoperiod for 10-14 days. Within 10 days the green shoots started to emerge. Two to five mm shoots were harvested and transferred to shoot elongation medium (as described above) containing cefotaxim, carbenicillin and kanamycin sulphate (25-125 mg/l). After one week in the shoot elongation medium the shoots were harvested and analyzed. All transformation experiments were repeated independently three times with 250 explants each time for each Agrobacterium strain.

Analysis of the transformed callus or shoots. Fourteen 18-day old transformed shoots and calli (the latter resulting from cotyledonary nodes and unable to produce shoots) were harvested from the shoot elongation medium and tested for GUS activity using histochemical GUS assay (Jefferson et al., 1987)

Root induction from the regenerated shoots. Non-transgenic shoots (two weeks old) from the shoot elongation medium were subjected to two different rooting procedures. Five 10 mm long shoots were harvested from the shoot elongation medium (SEM), the basal region was cut in order to remove any callus or necrotic tissue and the shoot ends were dipped either in a lawn of Agrobacterium rhizogenes (strain ARqua1) and transferred to root induction medium 1 (Farhaeus medium (Dazzo and Hubbell, 1982) solidified with 0.9-1.0% Gelrite) or directly transferred to root induction medium 2 (½ MS salts, Gamborg's B5 vitamins, 2.5% sucrose, 0.5-2.00 mg/l indole 3-butyric acid (IBA), agar 0.8%, pH 5.8) without any Agrobacterium rhizogenes inoculum. To prepare the A. rhizogenes lawn, a single colony was inoculated in 2 ml liquid Farhaeus medium containing 250 mg/l streptomycin in a 10 ml culture tube and cultured for 2-3 days at 28° C. on a rotary shaker. When the O.D. (600 nm) reached 1.0, 50-120 μl of the suspension was spread on a solid Farhaeus plate containing 250 mg/l streptomycin and 0.3% Gelrite. Within 3 days the bacteria formed a lawn on the plate which was subsequently used for infecting the shoot bases. Roots developed within 4 weeks and the rooted plants were transferred to the soil and grown in a growth chamber at 30° C. and 12 hrs of light.

Study of root anatomy. In order to analyze the root histology, 10-100 Mm thick sections were prepared from the roots or the junction of root and shoot, stained with 1% toluidine blue in water and observed under a stereo microscope (Olympus SZX 12).

### Example 2

### Results and Discussion

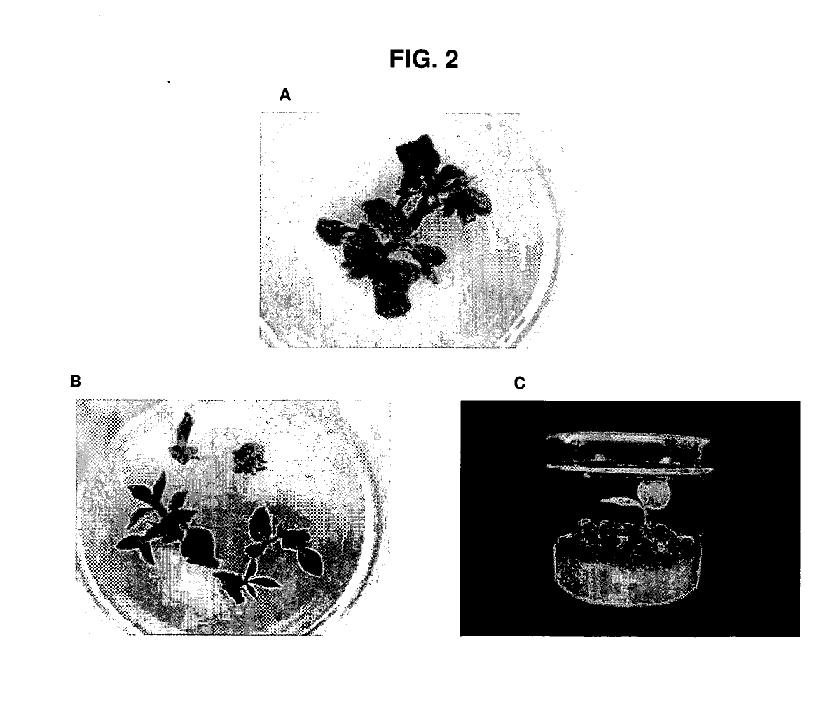

Multiple shoot bud initiation was observed in the cotyledonary node explants within 10 days in the shoot initiation medium (FIG. 1A). There were no significant differences between the genotypes Lewis and Kinman in regards to the morphology of shoot regeneration. After transferring the 5-10 mm long shoots from the shoot initiation medium to shoot elongation medium, a rapid proliferation in growth was noticed (FIGS. 1B-C). The shoot regeneration frequency in response to different growth regulators (Table 1) was different between the genotypes Kinman and Lewis, and BAP in conjunction with TDZ stimulated the highest level of shoot bud initiation and development. When IBA was used in combination with either BAP or TDZ, some of the regenerated plants showed noticeable variation in leaf and shoot morphology (data not shown). Within a span of 2-3 weeks shoot buds were initiated, differentiated and were ready for rooting (FIG. 1D and FIG. 2) in the case of both transformed and non-transformed shoots.

TABLE 1
Multiple shoot regeneration from the cotyledonary node explants
of two guar varieties (Kinman and Lewis) on B5 medium
containing different growth regulators

| Concentration of growth | *Shoot morphogenesis frequency | |
|---|---|---|
| in hormones (μM) | (%) in the genotype | |
| different shoot initiation media (SIM) | Kinman | Lewis |
| 1. TDZ 4.55 + BAP 4.44 | 42.37 | 41.91 |
| 2. TDZ 9.10 | 33.33 | 31.67 |
| 3. BAP 4.4 + NAA 5.37 | 14.01 | 14.22 |
| 4. BAP 4.44 | 26.49 | 25.89 |
| 5. BAP 4.44 + IBA 24.6 | 33.98 | 33.11 |
| 6. TDZ 4.55 + IAA 1.43 | 35.51 | 35.66 |
| 7. BAP 13.3 + IAA 11.4 | 31.94 | 32.43 |
| 8. BAP 4.44 + GA3 1.30 | 39.52 | 38.75 |

*Shoot morphogenesis frequency was calculated as the percentage of explants that formed shoots.

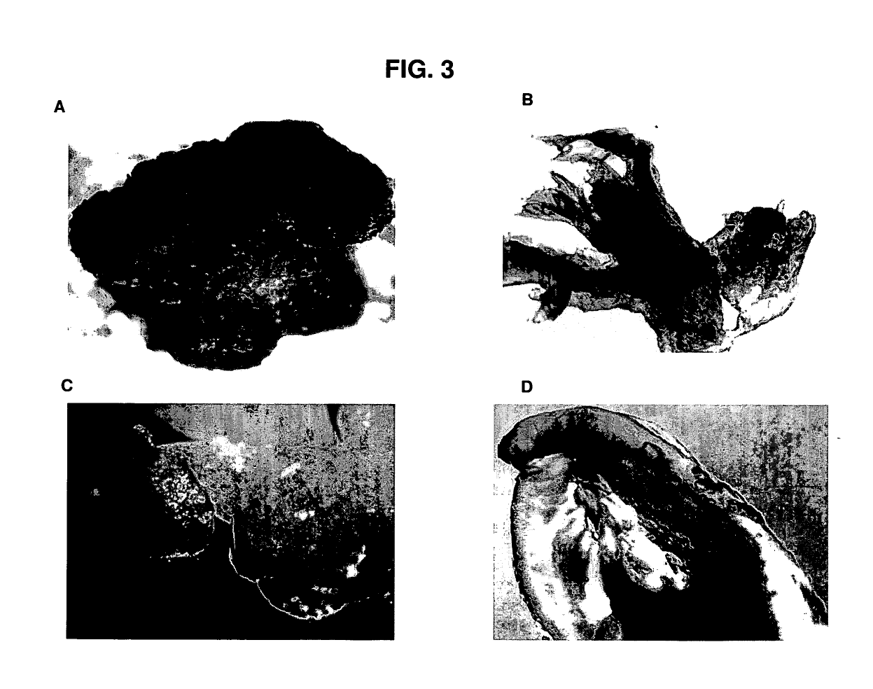

A protocol for the transformation of Guar has previously been published (Joersbo et al., 1999). In order to improve the transformation efficiency under our laboratory conditions, a number of parameters were altered. The infectivity and transformation efficiency of six Agrobacterium strains with four different genotypes of guar (data not shown) was studied and compared. The effect on transformation of five different Agrobacterium strains (Table 2) with guar cultivars Kinman and Lewis was evaluated by determining the percentage of viable GUS-positive shoots or calli (FIGS. 3A-D) regenerated per inoculated explant. As reported previously (Joersbo et al., 1999), the cultivar Lewis exhibited the highest transformation frequency of 1.09% (Table 2). The Agrobacterium strain C58C1 (Hamilton and Fall, 1971) gave the highest transformation efficiency (1.09%) for cultivar Lewis, followed by the efficiency of strain LBA 4404 (1.01%). Joersbo et al. (1999) reported that LBA 4404 gave the highest transformation efficiency (0.8%) in the cultivar Lewis (Joersbo et al., 1999).

TABLE 2
Effect of different Agrobacterium strains on transformation efficiency
in the cultivars Kinman and Lewis

Transformation efficiency (%) in

| Agrobacterium strain | Kinman | Lewis |
|---|---|---|
| EHA 105 | 0.71 | 0.74 |
| AGL1 | 0.89 | 0.91 |
| LBA4404 | 0.90 | 0.93 |
| C58C1 | 1.01 | 1.09 |
| GV3101 | 0.66 | 0.61 |

In the previously described guar transformation protocol (Joersbo et al., 1999), rooting of the transgenic shoots was accomplished by grafting on decapitated non-transgenic seedlings. American cultivars of Guar such as Kinman and Lewis are transformable but not easy to root in vitro. On the other hand, there has been no report of transformation in the Indian cultivars, e.g., HGS 365, HG 75, RGC 936 and CH 4-2, which are otherwise highly regenerable with the ability of rooting as reported previously (Prem et al., 2005). In an effort to induce rooting in regenerated guar shoots, IBA was used, as reported by Prem et al., and a combination of IBA and NAA. With only IBA more than 90% of the shoots formed callus at the base, but IBA in combination with NAA resulted in rooting in more than 80% of the shoots within four weeks. After another 3 weeks the plantlets were ready to be transferred to soil.

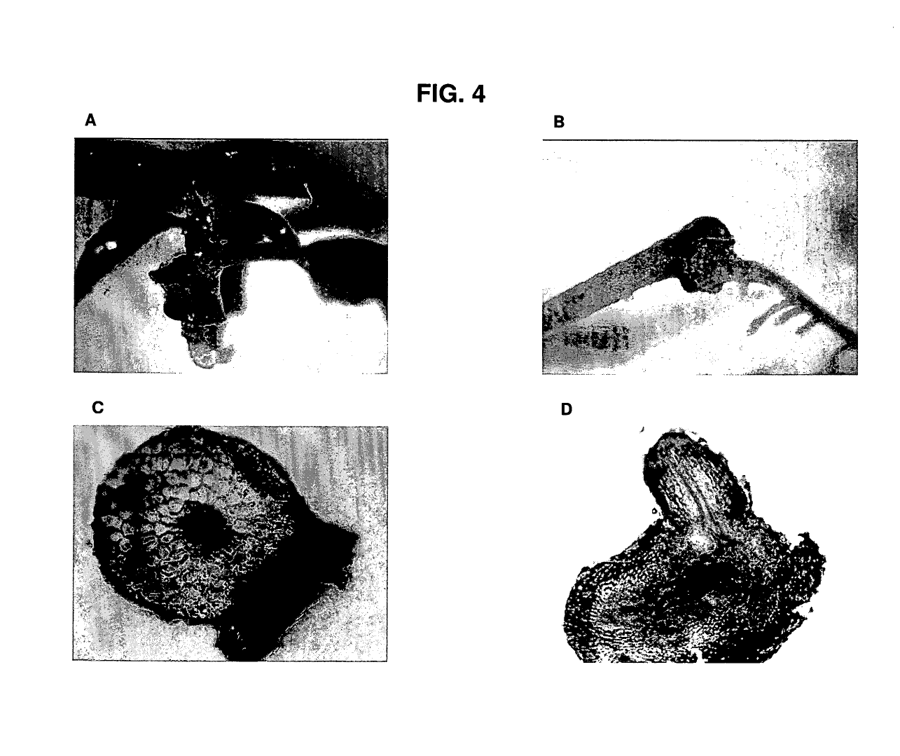

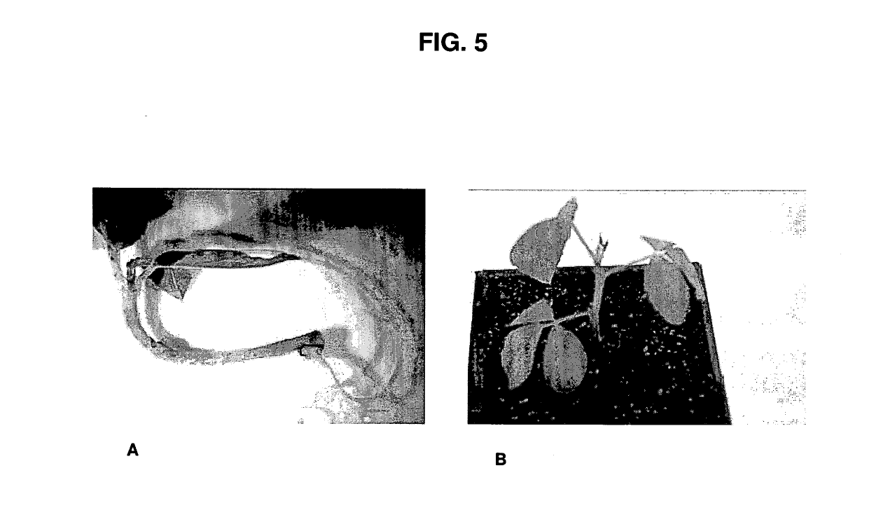

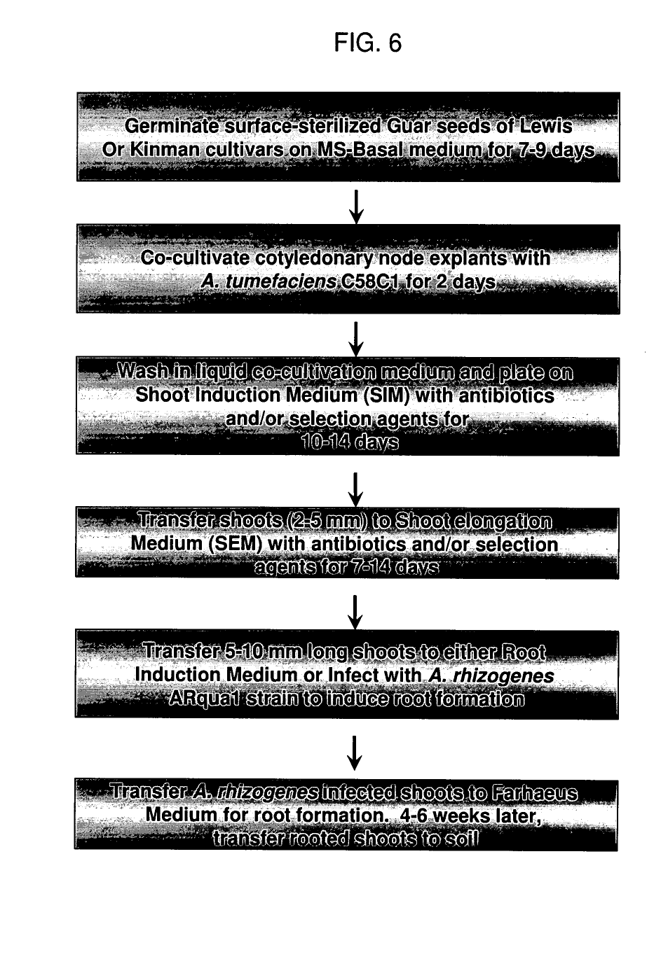

Agrobacterium rhizogenes has been used widely to induce rooting in many gymnosperms and angiosperms including almond (Damiano and Monticello, 1998), apple (Sutter and Luzza, 1993), kiwi (Rugini et al., 1991), walnut (Caboni et al., 1996), Pinus (McAfee et al., 1993) and Eucalyptus (MacRae and Van Staden, 1993). Here is reported for the first time that A. rhizogenes can induce rooting in Guar (FIGS. 4A-D and FIGS. 5A-B). In Table 3, A. rhizogenes mediated rooting is compared with the rooting induced by rooting hormones. Agrobacterium rhizogenes mediated roots have identical histology to wild-type roots, and have normal vascular connection with the stem. These roots are thus morphologically and anatomically normal and can support the normal growth of the plant in soil (FIGS. 5A-B).

TABLE 3
Comparison of rooting methods/media

| | | Number of days - | |
|---|---|---|---|
| | | from root bud | |
| | Rooting | initiation to | Root |
| Rooting method/ | efficiency* | hardening in the | morphology |
| Medium | (%) | green house | and anatomy |
| A. rhizogenes in | 35-45% | 4-6 weeks | Normal |
| Färhaeus medium | | | |
| MS salts, B5 vitamins, | 80% | 6-8 weeks | Normal |
| 2.5% sugar, IBA, NAA | | | |
| MS salts, B5 vitamins, | 0% | | |
| 2.5% sucrose, IBA | | | |

*Rooting efficiency was calculated as the percentage of total number of shoots forming root buds in the rooting medium.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The references listed below are incorporated herein by reference to the extent that they supplement, explain, provide a background for, or teach methodology, techniques, and/or compositions employed herein.

U.S. Pat. No. 3,710,511; U.S. Pat. No. 3,861,709; U.S. Pat. No. 4,535,060; U.S. Pat. No. 4,654,465; U.S. Pat. No. 4,727,219; U.S. Pat. No. 4,769,061; U.S. Pat. No. 4,795,855; U.S. Pat. No. 4,810,648; U.S. Pat. No. 4,940,835; U.S. Pat. No. 4,954,442; U.S. Pat. No. 4,975,374; U.S. Pat. No. 5,004,863; U.S. Pat. No. 5,159,135; U.S. Pat. No. 5,188,958; U.S. Pat. No. 5,262,316; U.S. Pat. No. 5,416,011; U.S. Pat. No. 5,463,174; U.S. Pat. No. 5,530,182; U.S. Pat. No. 5,530,191; U.S. Pat. No. 5,545,818; U.S. Pat. No. 5,550,318; U.S. Pat. No. 5,563,055; U.S. Pat. No. 5,565,347; U.S. Pat. No. 5,569,834; U.S. Pat. No. 5,589,615; U.S. Pat. No. 5,591,616; U.S. Pat. No. 5,610,042; U.S. Pat. No. 5,625,132; U.S. Pat. No. 5,684,242; U.S. Pat. No. 5,689,041; U.S. Pat. No. 5,689,053; U.S. Pat. No. 5,693,512; U.S. Pat. No. 5,712,112; U.S. Pat. No. 5,733,744; U.S. Pat. No. 5,741,684; U.S. Pat. No. 5,750,871; U.S. Pat. No. 5,824,872; U.S. Pat. No. 5,824,877; U.S. Pat. No. 5,846,797; U.S. Pat. No. 5,919,919; U.S. Pat. No. 5,922,928; U.S. Pat. No. 5,929,300; U.S. Pat. No. 5,932,782; U.S. Pat. No. 5,948,956; U.S. Pat. No. 5,952,543; U.S. Pat. No. 5,977,439; U.S. Pat. No. 5,981,840; U.S. Pat. No. 5,994,624; U.S. Pat. No. 6,037,522; U.S. Pat. No. 6,040,497; U.S. Pat. No. 6,040,498; U.S. Pat. No. 6,051,757; U.S. Pat. No. 6,074,876; U.S. Pat. No. 6,074,877; U.S. Pat. No. 6,103,955; U.S. Pat. No. 6,162,965; U.S. Pat. No. 6,215,051; U.S. Pat. No. 6,255,115; U.S. Pat. No. 6,255,559; U.S. Pat. No. 6,265,638; U.S. Pat. No. 6,274,791; U.S. Pat. No. 6,300,545; U.S. Pat. No. 6,307,127; U.S. Pat. No. 6,323,396; U.S. Pat. No. 6,369,298; U.S. Pat. No. 6,384,301; U.S. Pat. No. 6,420,630; U.S. Pat. No. 6,455,761; U.S. Pat. No. 6,603,061; U.S. Pat. No. 6,620,986; U.S. Pat. No. 6,664,108; U.S. Pat. No. 6,686,515; U.S. Pat. No. 6,696,622; U.S. Pat. No. 6,759,573; U.S. Pat. No. 6,800,791; U.S. Pat. No. 6,822,144; U.S. Pat. No. 6,846,971
Abe et al., J. Biol. Chem., 262:16793, 1987.
Arondel et al., Science, 258(5086):1353-1355 1992.
Bansal et al., J Physiol Res 7:57-60, 1994.
Beachy et al., Ann. Rev. Phytopathol., 28:451, 1990.
Beardmore et al., Physiol. Plant Pathol., 22:209-220, 1983.
Bevan et al., Nucleic Acids Res., 11(2):369-385, 1983.
Boudet et al., New Phytologist, 129:203-236, 1995.
Caboni, et al., Plant Science 118:203-208, 1996.
Callis et al., Genes Dev., 1:1183-1200, 1987.
Chandler et al., Plant Cell, 1:1175-1183, 1989.
Chu et al., Scientia Sinica, 18:659-668, 1975.
Conkling et al., Plant Physiol., 93:1203-1211, 1990.
Damiano and Monticelli, Electron. J. Biotechnol. 1:12-13, 1998.
Dazzo and Hubbell, In: Control of Root Hair Infection, Vol 2, Oxford, Oxford University Press, 1982.
DE Appln. 3642 829
De Block et al., EMBO J., 6(9):2513-2518, 1987.
De Block et al., Plant Physiol., 91:694-701, 1989.
DeGreef et al., Bio/Technology, 7:61, 1989.
Dellaporta et al., In: Chromosome Structure and Function: Impact of New Concepts, 18th Stadler Genetics Symposium, 11:263-282, 1988.
Dixon et al., Gene, 179:61-71. 1996.
Ebert et al., Proc. Natl. Acad. Sci. USA, 84:5745-5749, 1987.
Elliot et al., Plant Molec. Biol., 21:515, 1993.
European Appln. 0 242 246
European Appln. 0 333 033
European Appln. 0616644
European Appln. 154 204
Fisher et al., Plant Physiol., 102:1045, 1993.
Fox et al. Proc. Natl. Acad. Sci. USA, 90(6):2486-2490, 1993.
Fraley et al., Bio/Technology, 3:629-635, 1985.
Fromm et al., Nature, 319(6056):791-793, 1986.
Gallie et al., The Plant Cell, 1:301-311, 1989.
Gamborg, et al., Exp. Cell Research, 50:151-158, 1968.
Geiser et al., Gene, 48:109, 1986.
Gleen et al., Plant Molec. Biology, 18:1185-1187, 1992.
Hamilton and Fall, Experientia, 27:229-230, 1971.
Hammock et al., Nature, 344:458, 1990.
Hanna and Elsner, Crop Sci., 39:1258, 1999.
Hartman et al., Bio/Technology, 12:919-923, 1994.
Haseloff et al., Proc. Natl. Acad. Sci. USA, 94:2122-2127, 1997.
Hayes et al., Biochem. J., 285(Pt 1):173-180, 1992.
Hiei et al., Plant Mol. Biol., 35(1-2):205-218, 1997.
Holsters, et al., Plasmid, 3:212-230, 1980.
Hudspeth and Grula, Plant Mol. Biol., 12:579-589, 1989.
Huub et al., Plant Molec. Biol., 21:985, 1993.
Ikuta et al., Bio/technol., 8:241-242, 1990.
Intrieri and Buiatti, Molecular Phylogenetics and Evolution, 20:100-110, 2001.
Ishida et al., Nat. Biotechnol., 14:745-750, 1996.
Jackson and Doughton, In: Guar: A Potential Industrial Crop for the Dry Tropics of Australia, The Australia Inst. of Agric. Science, 1982.
Jefferson et al., EMBO J., 6:3901-3907, 1987.
Joersbo et al., Mol. Breeding, 5:521-529, 1999.
Joersbo et al., Mol. Breeding, 7:211-219, 2001.
Jones et al., Science, 266:7891, 1994.
Katz et al., J. Gen. Microbiol., 129:2703-2714, 1983.
Kirihara et al., Gene, 71(2):359-370, 1988.
Klee et al., BioTechnology, 3(7):637-642, 1985.
Knutzon et al., Proc. Natl. Acad. Sci. USA, 89:2624, 1992.
Lawton et al., Plant Mol. Biol. 9:315-324, 1987.
Lee et al., EMBO J., 7:1241, 1988.
Llewellyn et al., J. Mol. Biol., 195(1):115-23, 1987.
Logemann et al., Biotechnology, 10:305, 1992.
MacRae and Van Staden, Tree Physiology, 12:411-418, 1993.
Marshall et al., Theor. Appl. Genet., 83:4:35, 1992.
Martin et al., Science, 262:1432, 1993.
McAfee et al., Plant Cell Tiss. Organ Cult., 34:53-62, 1993.
McCormac et al., Euphytica, 99(1):17-25, 1998.
McDonough et al., J. Biol. Chem., 267(9):5931-5936, 1992.
Miki et al., Theor. Appl. Genet., 80:449, 1990.
Mindrinos et al., Cell, 78(6):1089-1099, 1994.
Murakami et al., Mol. Gen. Genet., 205:42-50, 1986.
Murashige and Skoog, Physiol. Plant, 15:473-497, 1962.
Odell et al., Nature, 313:810-812, 1985.
Ogawa et al., Sci. Rep., 13:42-48, 1973.
Otani et al., Plant Science, 94:151-159, 1993.
Ow et al., Science, 234:856-859, 1986.
PCT Appln. US 93/06487
PCT Appln. WO 91/13972
PCT Appln. WO 97/4103
PCT Appln. WO 97/41228
Pen et al., Biotechnology, 10:292, 1992.
Popelka et al., Plant Cell Rep 25:304-312, 2006.
Potrykus et al., Mol. Gen. Genet., 199(2):169-177, 1985.
Potrykus et al., Mol. Gen. Genet., 199:183-188, 1985.
Prasher et al., Biochem. Biophys. Res. Commun., 126(3):1259-1268, 1985.
Prem et al., Plant Cell 80:209-214, 2005.
Przibila et al., Plant Cell, 3:169, 1991.
Raboy et al., Plant Physiol., 124(1):355-368.
Rae et al., Australian J. Plant Physiol., 28:289-297, 2001.
Ramulu and Rao, J. Physiol. Res., 20:7-9, 1993a.
Ramulu and Rao, J. Physiol. Res., 6:71-72, 1993b.
Reddy et al., Plant Mol. Biol., 22(2):293-300, 1993.
Reichel et al., Proc. Natl. Acad. Sci. USA, 93(12):5888-5893, 1996.
Rogers et al., Methods Enzymol., 153:253-277, 1987.
Rugini et al., Plant Cell Reports, 10:291-295, 1991.
Sainy and Paroda, In; Guar cultivation in Haryana, Hisar Haryana, India, Department of Plant Breeding, Chaudhary Charan Singh Haryana Agricultural University, 1984.
Saxena and Gill, Biol. Plant, 28:313-315, 1986.
Saxena et al., Plant Cell Tiss. Organ Cult., 6:173-176, 1986.
Sergaard et al., J. Biol. Chem., 268:22480, 1993.
Sheen et al., Plant J., 8(5):777-784, 1995.
Shiroza et al., J. Bacteol., 170:810, 1988.
Spangenberg et al., In: Biotechnology in forage and turf grass improvement, Springer, Berlin, 1998.
Spencer et al., Plant Mol. Biol., 18(2):201-210, 1992.
Stafford, Lewis—a New Guar Variety, Texas Agric. Exp. Sta. Bull. L-2177, Texas A&M Univ. Sys., College Station, Texas, 1986.
Stalker et al., Science, 242:419-422, 1988.
Steinmetz et al., Mol. Gen. Genet., 20:220, 1985.
Sullivan et al., Mol. Gen. Genet., 215(3):431-440, 1989.
Sumitani et al., Biosci. Biotech. Biochem., 57:1243, 1993.
Sutcliffe, Proc. Natl. Acad. Sci. USA, 75:3737-3741, 1978.
Sutter and Luzza, Int. J. Plant Sci., 154:59-67, 1993.
Tavladoraki et al., Nature, 366:469, 1993.
Taylor et al., Seventh Int'l Symposium on Molecular Plant-Microbe Interactions, Edinburgh, Scotland, Abstract W97, 1994.
Texas Agricultural Experiment Station, Kinman and Esser, New Guar Varieties. Bull. L-1356. Texas Agricultural Experiment Station, Texas A&M University System, College Station, 1975.
Thillet et al., J. Biol. Chem., 263:12500-12508, 1988.
Thomas et al., Plant Sci., 69:189-198, 1990.
Thompson et al., EMBO J., 6(9):2519-2523, 1987.
Tian et al., Genes Dev., 11(1):72-82, 1997.
Tingay et al., Plant J., 11(6):1369-1376, 1997.
Tripp et al., Keys to Profitable Guar Production, Texas Agric. Exten. Serv. Bull. B-1399, Texas A&M Univ. Sys., College Station, Texas.
Twell et al., Plant Physiol., 91:1270-1274, 1989.
Undersander, et al., Guar, In: Alternative Field Crops Manual, Univ. Wisconsin-Extension, Cooperative Extension; University of Minnesota, Center for Alternative Plant & Animal Products; Minnesota Extension Service, Madison, Wis., 1991.
Van Damme et al., Plant Molec. Biol., 24:25, 1994.
Van de Velde et al., Plant Science, 165:1281-1288, 2003.
Van Hartingsveldt et al., Gene, 127:87, 1993.
Vasil et al., Plant Physiol., 91:1575-1579, 1989.
Wang et al., Molec. Cell. Biol., 12(8):3399-3406, 1992.
Whistler and Hymowitz, In: Guar: Production, Nutrition and Industrial Use, Purdue Univ. Press, Lafayette, Indiana, 1979.
Yang and Russell, Proc. Natl. Acad. Sci. USA, 87:4144-4148, 1990. Zukowsky et al., Proc. Natl. Acad. Sci. USA, 80:1101-1105, 1983.

What is claimed is:

1. A method of transforming a guar plant comprising:
(a) obtaining an explant from a guar seedling;
(b) contacting the explant with a recombinant Agrobacterium strain containing a DNA of interest to transform at least a first cell of the explant with the DNA of interest;
(c) culturing the explant to induce formation of roots;
(d) allowing roots to form from the explant; and
(e) cultivating the explant under plant growth conditions to produce a transgenic guar plant comprising the DNA of interest.

2. The method of claim 1, wherein the explant is a cotyledon explant.

3. The method of claim 1, wherein the explant comprises a cotyledonary node.

4. The method of claim 1, wherein the DNA of interest comprises a selectable marker and the explant is contacted with a selective agent following transforming the first cell with the DNA of interest.

5. The method of claim 1, wherein said guar seed is a seed of the cultivar Lewis or Kinman.

6. The method of claim 1, wherein the Agrobacterium tumefaciens strain is strain C58C1.

7. The method of claim 1, wherein culturing the explant to induce formation of roots comprises contacting the explant with Shoot Initiation Medium (SIM).

8. The method of claim 7, wherein culturing the explant to induce formation of roots comprises contacting the explant with Shoot Elongation Medium (SEM) and allowing shoots to form.

9. The method of claim 8, wherein the shoots are about 2-5 mm in length.

10. The method of claim 8, wherein the shoots are about 5-10 mm in length.

11. The method of claim 1, wherein culturing the explant to induce formation of roots comprises infection with A. rhizogenes or culturing in root induction medium.

12. The method of claim 1, wherein culturing the explant to induce formation of roots comprises culturing in Farhaeus medium for about 4 to 6 weeks.

13. The method of claim 12, wherein cultivating the explant under plant growth conditions comprises transferring the explant to soil.

14. The method of claim 11, wherein the A. rhizogenes is strain ARqua1.

15. The method of claim 1, wherein the explant is obtained from about 7 to 9 days post-germination.

16. The method of claim 1, wherein step (b) comprises co-cultivating the explant and Agrobacterium strain for about 2 days.

17. The method of claim 7, wherein the explant is contacted with the Shoot Initiation Medium for about 10 to 14 days.

18. The method of claim 17, wherein the Shoot Initiation Medium comprises a selective agent.

19. The method of claim 8, wherein the explant is contacted with the Shoot Elongation Medium (SEM) for about 7 to 14 days.

20. The method of claim 1, wherein culturing the explant to induce formation of roots comprises culturing the explant in Root Induction Medium 1 and/or Root Induction Medium 2.

21. The method of claim 1, wherein the DNA of interest confers a trait selected from the group consisting of herbicide tolerance, an insect resistance, disease resistance, pest resistance, improved nutritional quality, modified carbohydrate metabolism and modified lipid metabolism.

22. The method of claim 1, wherein the DNA of interest encodes β-mannan synthase or α-galactosyltransferase.

23. A guar plant generated according to the method of claim 1.

24. A progeny of the plant of claim 23.

25. A plant part of the plant of claim 23.

26. A cell of the plant of claim 23.

27. A seed of the plant of claim 23.

28. A method of producing food or feed, comprising (a) obtaining the plant of claim 23 or a part thereof; and (b) producing food or feed from the plant or part thereof.

29. A method of breeding a plant comprising (a) obtaining the plant of claim 23; and (b) breeding said plant with a second guar plant.
